(19) United States
(12) Patent Application Publication
(10) Pub. No.: US 2025/0381387 A1
(43) Pub. Date: Dec. 18, 2025

(54) SYSTEM FOR PREDICTION OF MEDICAL DEVICE STARTUP CONDITION

(71) Applicant: BOSTON SCIENTIFIC SCIMED, INC., MAPLE GROVE, MN (US)

(72) Inventors: Veronica Ramos, Miami, FL (US); Jennifer Carrasquilla, Miami Lakes, FL (US); Janny Garcia, Miami Springs, FL (US)

(73) Assignee: BOSTON SCIENTIFIC SCIMED, INC., MAPLE GROVE, MN (US)

(21) Appl. No.: 19/237,822

(22) Filed: Jun. 13, 2025

Related U.S. Application Data
(60) Provisional application No. 63/660,147, filed on Jun. 14, 2024.

(51) Int. Cl. A61M 60/508; A61M 60/178; A61M 60/216; A61M 60/585; A61M 60/804; G05B 13/02; G16H 40/67

(52) U.S. Cl. CPC A61M 60/508; A61M 60/178; A61M 60/216; A61M 60/585; A61M 60/804; G05B 13/0265; G16H 40/67; A61M 2205/18; A61M 2205/3365

(57) ABSTRACT

A system comprising: an implantable medical device (IMD) configured to determine values for one or more parameters associated with a startup of the IMD; and a programming device comprising: communications circuitry; memory; a user interface (UI); and processing circuitry configured to: retrieve, from the IMD and via the communications circuitry, the parameter values for the one or more parameters; apply a machine learning (ML) model stored in the memory to the parameter values, wherein the ML model is trained via a data set comprising a plurality of parameter values from one or more IMDs and corresponding results of startup procedures performed by the one or more IMDs; determine, based on outputs from the ML model, a probability of the IMD having a future startup failure within a period of time; and cause the UI to output a notification indicating the probability.

### CROSS-REFERENCE TO RELATED APPLICATION

This application claims the benefit of U.S. Provisional Application Ser. No. 63/660,147, filed Jun. 14, 2024, entitled “SYSTEM FOR PREDICTION OF MEDICAL DEVICE STARTUP CONDITION”, which is incorporated by reference herein in its entirety.

### TECHNICAL FIELD

This disclosure relates to medical device systems and, more particularly, to medical device systems for monitoring startup behavior of an implantable medical device

### BACKGROUND

After a medical device is implanted within a body of a patient, a medical device system may perform a startup procedure to power on the medical device. The medical device may need to be powered on after implantation, after replacement, after reconnection of the medical device to a power source, or the like.

### SUMMARY

The devices, systems, and techniques of this disclosure generally relate to prediction of startup condition(s) of an implantable medical device (IMD). An example IMD may be required to initiate a startup procedure through the lifespan of the IMD. After a successful completion of a startup procedure, the IMD may power on and sense physiological information from and/or deliver medical therapy to the body of the patient. The failure by the IMD to complete the startup procedure may result in an inability of the IMD to successfully sense physiological information from and/or deliver medical therapy to the body of the patient.

The devices, systems, and techniques of this disclosure may provide one or more techniques to predict a probability that the IMD will successfully perform a startup procedure. The techniques described herein may include the application of an algorithm (e.g., a machine learning (ML) technique) to determine the probability of a successful startup condition based on parameter data stored in the IMD. The devices, systems, and techniques described herein may allow for the successfully prediction of startup conditions of an example IMD without requiring extensive monitoring of the operations of the IMD and/or removal of the IMD from within the body of the patient. The devices, systems, and techniques described herein may be used to evaluate the startup conditions of already-implanted IMDs without requiring modification and/or removal of the IMDs. Prediction of startup conditions of the IMD prior to IMD performing a startup procedure may allow a clinician to examine, repair, and/or replace the IMD before the IMD fails to complete a startup condition.

In some examples, the IMD may be a blood pump (e.g., a ventricular assist device (VAD)). In such examples, a startup failure of the blood pump may lead to an inability for the blood pump to provide vascular circulation support, which may lead to adverse effects on patient health. The devices, systems, and techniques described herein applies the algorithm to parameter data stored by the blood pump to determine a likelihood of startup failure by the blood pump during an upcoming startup procedure. The different parameters inputted into the algorithm may be independent and/or unrelated to each other, which makes it infeasible for a clinician to mentally determine the likelihood of startup failure by the blood pump based purely on parameter values. Application of the algorithm to the parameter data stored by the blood pump provides for a determination of the likelihood that the blood pump will experience a startup failure. The determined likelihood may provide a warning to the clinician to replace the blood pump within the patient and/or to the blood pump to adjust the startup procedure, e.g., to reduce or eliminate a risk of actual startup failure.

In some examples, the disclosure describes a system comprising: an implantable medical device (IMD) comprising: an impeller configured to cause blood flow in a patient; a first memory; parameter sampling circuitry configured to determine values for one or more parameters associated with a startup of the implantable blood pump; and first processing circuitry configured to store, in the first memory, the values for one or more parameters with corresponding timestamps; and a programming device comprising: communications circuitry; a second memory; a user interface (UI); and processing circuitry configured to: retrieve, from the IMD and via the communications circuitry, the values for the one or more parameters associated with the startup of the IMD; apply a machine learning (ML) model stored in the memory to the values for the one or more parameters, wherein the ML model is trained via a data set comprising a plurality of parameter values from one or more IMDs and corresponding results of startup procedures performed by the one or more IMDs; determine, based on an output from the ML model, a probability of the IMD having a future startup failure within a period of time, wherein blood is not pumped by the IMD after an occurrence of a startup failure; and cause the UI to output a notification indicating the probability of the IMD experiencing the future startup failure.

In some examples, this disclosure describes a computing device comprising: communications circuitry; memory; a user interface (UI); and processing circuitry configured to: retrieve, via communications circuitry and from an implantable medical device (IMD) implanted in a patient, values for one or more parameters associated with a startup of the IMD; apply a machine learning (ML) model stored in the memory to the parameter values, wherein the ML model is trained via a data set comprising a plurality of parameter values from one or more IMDs and corresponding results of startup procedures performed by the one or more IMDs; determine, based on outputs from the ML model, a probability of the IMD having a future startup failure within a period of time; and cause the UI to output a notification indicating the probability of the IMD experiencing a future startup failure.

In some examples, this disclosure describes a computer-readable medium comprising instructions that, when executed, causes processing circuitry of a computing device to: retrieve, via communications circuitry of the computing device and from an implantable medical device (IMD) implanted in a patient, values for one or more parameters associated with a startup of the IMD; apply a machine learning (ML) model to the parameter values, wherein the ML model is trained via a data set comprising a plurality of parameter values from one or more IMDs and corresponding results of startup procedures performed by the one or more IMDs; determine, based on outputs from the ML model, a probability of the IMD having a future startup failure within a period of time; and cause a user interface (UI) of the computing device to output a notification indicating the probability of the IMD experiencing a future startup failure within the period of time.

In some examples, this disclosure describes a system comprising: an implantable blood pump comprising: a pump housing; an impeller disposed within the pump housing, and a motor disposed within the pump housing and coupled to the impeller, wherein the motor is configured to rotate the impeller within the pump housing to pump blood through the implantable blood pump; a power supply; and a controller coupled to the implantable blood pump and to the power supply, the controller comprising: a user interface (UI), a memory, and a processing circuitry configured to: cause the power supply to transmit an electrical signal to the motor of the implantable blood pump to start the implantable blood pump; retrieve from one or more of the impeller, the motor, or the power supply, values for the one or more parameters; apply a machine learning (ML) model stored in the memory to the values for the one or more parameters, wherein the ML model is trained via a data set comprising a plurality of parameter values from one or more implantable blood pumps and corresponding results of startup procedures performed by the one or more implantable blood pumps; determine, based on an output from the ML model, a probability of the implantable blood pump having a future startup failure within a period of time, wherein blood is not pumped by the implantable blood pump after an occurrence of a startup failure; and cause the UI to output a notification indicating the probability of the implantable blood pump experiencing the future startup failure.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram of a medical device system for predicting startup conditions of an implantable medical device (IMD).

FIG. 2 is a block diagram illustrating an example configuration of an example implantable medical device (IMD) of the system of FIG. 1.

FIG. 3 is a conceptual diagram illustrating an example neural network configured to predict the startup conditions of the IMD.

FIG. 4 is a conceptual diagram illustrating an example process of inputting data into an example model for the prediction of the startup conditions of the IMD.

FIG. 5 is a block diagram illustrating an example process of training an example model for the prediction of the startup conditions of the IMD.

FIG. 6 is a flowchart illustrating an example process of predicting the startup conditions of the IMD.

FIG. 7 is a flowchart illustrating another example process of predicting and outputting the startup conditions of the IMD.

FIG. 8 is a conceptual diagram of a medical device system for predicting startup conditions of an implantable blood pump.

### DETAILED DESCRIPTION

A medical device system may include an implantable medical device (IMD) implanted within a body of a patient. The IMD may sense physiological information from and/or deliver medical therapy to the body of the patient. The IMD may be in communication with an external computing device (e.g., external programmer) of the medical device system. In some examples, the IMD is coupled to one or more external power sources.

During the operational lifespan of the IMD, the medical device system may need to power on the IMD and/or one or more subsystems of the IMD via a startup procedure on one or more occasions. During the startup procedure, the medical device system may supply power to the IMD (e.g., via a power source internal to the IMD, via an implanted power source coupled to the IMD, via an external power source) to cause one or more components of the IMD to power on from an OFF state to an ON state. The medical device system may need to power on the IMD, e.g., after implantation of the IMD, after replacement of one or more components of the IMD, after replacement of an external controller of the IMD, after replacement of an external power source to the IMD, after reconnecting the external power source to the IMD, after a decision by the patient and/or a clinician to power on the IMD, or the like.

When medical device system initiates the startup procedure, there is a possibility that the IMD will fail to power on after one or more attempts through the startup procedure, i.e., fail to complete the startup procedure. In such instances, the IMD may be incapable of sensing information from the body of the patient and/or deliver medical therapy to the patient. This may render the IMD at least temporarily incapable of performing the IMD's intended function and/or may require the patient to visit a clinician to repair and/or replace the IMD. In some examples, the IMD may experience one or more unsuccessful startup attempts and/or failure(s) to start within a time window (e.g., within an expected lifespan of the IMD).

The devices, systems, and techniques described herein predict a likelihood that the IMD will not complete a startup procedure (e.g., will fail to complete the startup procedure) based on information stored in the IMD. Prediction of the likelihood that the IMD will or will not complete the startup procedure prior to having the IMD undergo the startup procedure may allow the patient and/or the clinician to mitigate the possibility of a failure to start the IMD, e.g., by replacing the IMD, by adjusting parameters of the startup procedure, or the like. The techniques of this disclosure may be of particular advantage to systems, such as ventricular assist devices and other internal pumps, that use external batteries or power supplies to provide power to implanted medical devices. Such systems typically require frequent startup of the internal medical devices necessitated by swapping power supplies, e.g., changing batteries or transitioning between wall power and battery power.

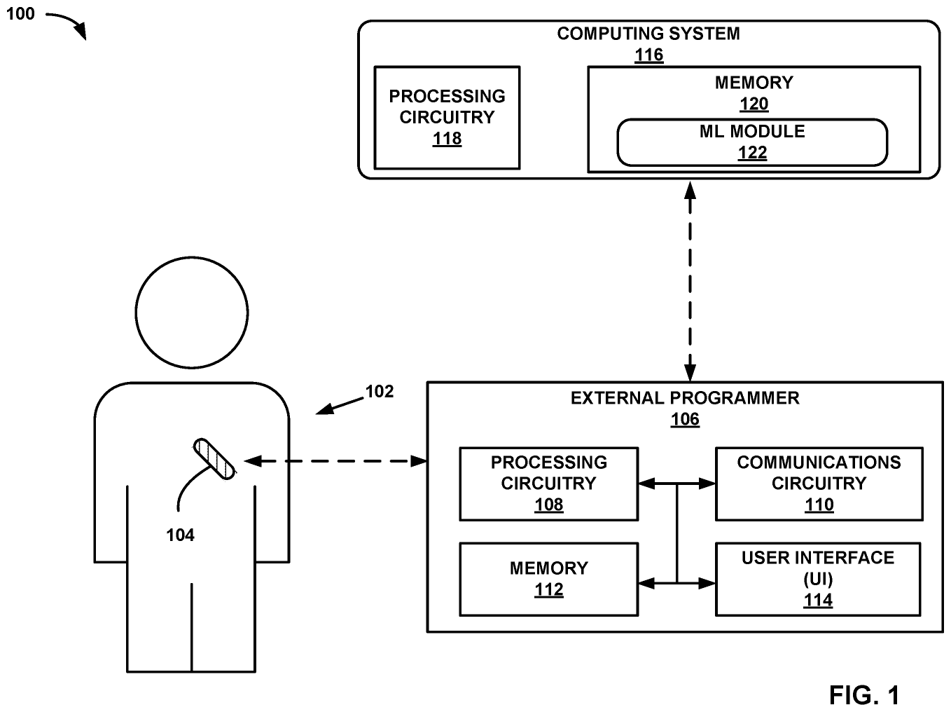

FIG. 1 is a conceptual diagram of a medical device system 100 (hereinafter “system 100”) for predicting startup conditions of an implantable medical device (IMD) 104. The IMD 104 may be implanted within a body of a patient 102. While IMD 104 is primarily described herein with regard to a ventricular assist device (VAD), IMD 104 may include other IMDs, including, but are not limited to, implantable cardioverter defibrillators (ICDs), insertable cardiac monitors (ICMs), or the like.

IMD 104 may be in communication (e.g., wired communication, wireless communication) with an external programmer 106. External programmer 106 may include components including, but is not limited to, processing circuitry 108, communications circuitry 110, memory 112, and user interface (UI) 114. External programmer 106 may be in communication with computing system 116. Computing system 116 may include one or more computing devices, computing systems, and/or cloud computing environments. Computing system 116 may include processing circuitry 118 and memory 120. Memory 120 may include machine learning (ML) module 122.

IMD 104 may store parameter values within the memory of IMD 104. At least a portion of the parameter values may correspond to prior startup attempts by IMD 104. Prior startup attempts may include successful and unsuccessful startup attempts by IMD 104. Parameters may include, but is not limited to, one or more of a number of startup attempts for each successful startup attempt for IMD 104, a number of prior unsuccessful startup attempts by IMD 104, a device identifier for IMD 104, a device type for IMD 104, an identifier for patient 102, a timestamp of each startup attempt by IMD 104, a duration of each startup attempt by IMD 104, an amplitude of each electrical current delivered to IMD 104 during startup attempts, or a voltage of each electrical current delivered to IMD 104 during startup attempts.

Processing circuitry 108 of external programmer 106 may retrieve at least a portion of the stored parameter values from IMD 104 via communications circuitry 110. Processing circuitry 108 may retrieve, from memory 120 of computing system 116 (e.g., from ML module 122), one or more modules and/or algorithms. The one or more modules and/or algorithms may include, but is not limited to, one or more machine learning (ML) models and/or algorithms or one or more rule-based models and/or algorithms. Processing circuitry 108 may store the retrieved parameter values and/or retrieved modules and/or algorithms in memory 112. The one or more model and/or algorithms may include rule-based expert systems and/or trained ML models. In some examples, the one or more models may include a rules-based expert system and processing circuitry 108 may determine rules for the one or more rule-based expert system, e.g., through training using machine learning techniques. In some examples, the one or more models may include one or more trained ML models, and processing circuitry 202 may define and train the one or more trained ML models using machine learning techniques. In some examples, processing circuitry 202 may automatically define the entirety of a trained ML model through training using machine learning techniques. In some examples, the trained ML model may not include any individual rules. Example machine learning techniques may include, but are not limited to, supervised learning, and semi-supervised learning. In some examples, processing circuitry 202 may train the one or more models using one or more algorithms including, but are not limited to, Bayesian algorithms, Clustering algorithms, decision-tree algorithms, regularization algorithms, regression algorithms, instance-based algorithms, artificial neural network algorithms, deep learning algorithms, or dimensionality reduction algorithms.

Processing circuitry 108 may input at least a portion of the retrieved parameter values into the one or more modules and/or algorithms to determine a likelihood that IMD 104 will experience an unsuccessful startup attempt and/or a failure to start. Processing circuitry 108 may cause UI 114 to output the determination to patient 102 and/or a clinician. In some examples, based on the determination, processing circuitry 108 transmits, via communications circuitry 110, instructions to IMD 104 to adjust one or more parameters of the startup procedure, e.g., to decrease a likelihood that IMD 104 will experience an unsuccessful startup attempt and/or a failure to start.

One or more of processing circuitry 108 or processing circuitry 118 may train the one or more models and/or algorithms based on a training data set including a plurality of startup attempts (e.g., both successful and unsuccessful startup attempts) and corresponding parameter values. The training data may be retrieved from IMD 104 and/or one or more other IMDs.

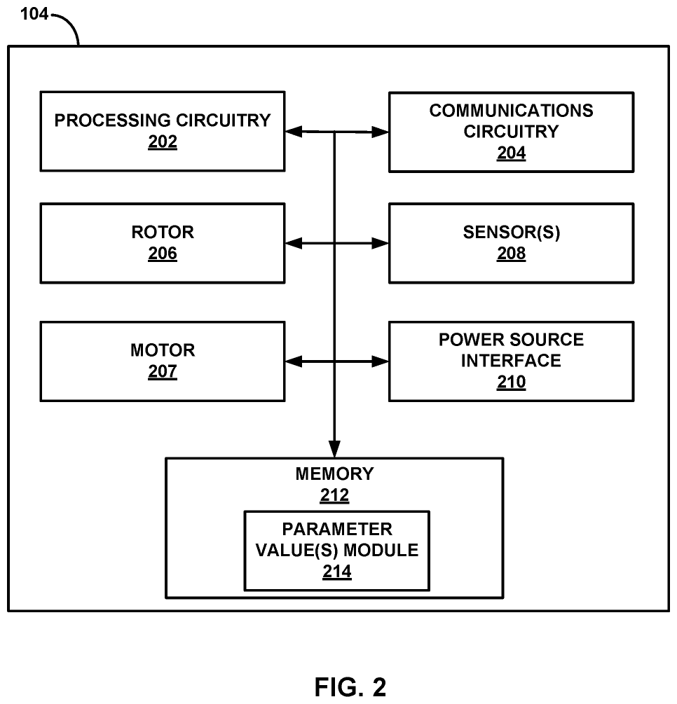

FIG. 2 is a block diagram illustrating an example configuration of an example IMD 104 of system 100 of FIG. 1. IMD 104 may be an implanted cardiac device including, but not limited to, a VAD, an ICM, an implantable pulse generator (IPG), an ICD, a cardiac resynchronization therapy (CRT) device, a drug pump, or the like. In the example shown in FIG. 2, which is a VAD, IMD 104 includes processing circuitry 202, communications circuitry 204, rotor 206, motor 207 sensor(s) 208, a power source interface 210, and memory 212. Memory 212 may include a parameter value(s) module 214. The various circuitry may be, or include, programmable or fixed function circuitry configured to perform the functions attributed to respective circuitry.

Memory 212 may store computer-readable instructions that, when executed by processing circuitry 202, cause IMD 104 to perform various functions. Memory 212 may be a storage device or other non-transitory medium. Memory 212 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Processing circuitry 202 may cause rotor 206 to rotate within IMD 104 to cause a fluid to flow through IMD 104, e.g., to assist the flow of blood within a heart of patient 102. Rotor 206 may be connected to an inlet and an outlet of IMD 104 (not pictured) and may be in fluid communication with one or more chambers of the heart of patient 102 and/or a blood vessel of patient 102 (e.g., an aorta of patient 102). Processing circuitry 202 may cause motor 207 to rotate rotor 206 to cause blood to flow through IMD 104 from the inlet to the outlet. The rotational speed of rotor 206 may be controlled by processing circuitry 202 and may vary over time, e.g., based on the cardiac cycle of patient 102. An example IMD 104 is described in commonly-owned U.S. patent application Ser. No. 17/454,954, filed Nov. 15, 2021, and entitled “Mechanical Circulatory Support Device,” the entire contents of which is incorporated herein by reference. Rotor 206 may include, but is not limited to, an impeller.

Power source interface 210 may electrically couple an external power source and/or an implantable power source external to IMD 104 to one or more components of IMD 104 (e.g., to processing circuitry 202, rotor 206, motor 207, etc.). Power source interface 210 may include a driveline configured to provide electrical and/or mechanical power from the external power source to IMD 104.

Processing circuitry 202 may include any one or more of a microprocessor, a controller, a digital signal processor (DSP), an application-specific integrated circuit (ASIC), a field-programmable gate array (FPGA), discrete logic circuitry, or any other processing circuitry configured to provide the functions attributed to processing circuitry 202 herein and may be embodied as firmware, hardware, software, or any combination thereof.

Processing circuitry 202 may initiate a startup procedure in response to receiving electrical signals and/or mechanical signals through power source interface 210. In some examples, processing circuitry 202 may initiate a startup procedure in response to receiving a startup instruction from external programmer 106 via communications circuitry 204. During the startup procedure, processing circuitry 202 may record and/or cause sensor(s) 208 to record values for a plurality of parameter during each attempt of the startup procedure. Processing circuitry 202 may store the recorded parameter values in parameter value(s) module 214 of memory 212. In some examples, processing circuitry 202 may update the stored parameter values based on instructions received form external programmer 106 and/or new values recorded by processing circuitry 202 during subsequent startup procedures.

Sensor(s) 208 may include one or more sensing elements that transduce patient physiological activity (e.g., patient cardiac activity) to an electrical signal to sense values of a respective patient parameter. Sensor(s) 208 may include one or more accelerometers, optical sensors, chemical sensors, temperature sensors, pressure sensors, or any other types of sensors. Sensor(s) 208 may include one or more sensing elements disposed within IMD 104 and configured to sense values of one or more functions of IMD 104 over time (e.g., whether one or more components of IMD 104 has been powered on, an amplitude and/or voltage of an electrical current received by IMD 104).

Communication circuitry 204 supports wireless communication between IMD 104 and external programmer 106. Processing circuitry 202 of IMD 104 may receive, from external programmer 106 and via communication circuitry 204, instructions to transmit stored parameter values to external programmer 106. In some examples, processing circuitry 202 automatically transmits stored parameter values to external programmer 106 via communications circuitry 204. Communication circuitry 204 may communicate with external programmer 106 via wired communication or by wireless communication techniques. Wireless communication techniques may include radiofrequency (RF) communication techniques, e.g., via an antenna (not shown). Communication circuitry 204 may include a radio transceiver configured for communication according to standards of protocols, such as 3G, 4G, 5G, WiFi (e.g., 802.11 or 802.15 ZigBee), Bluetooth®, or Bluetooth® Low Energy (BLE).

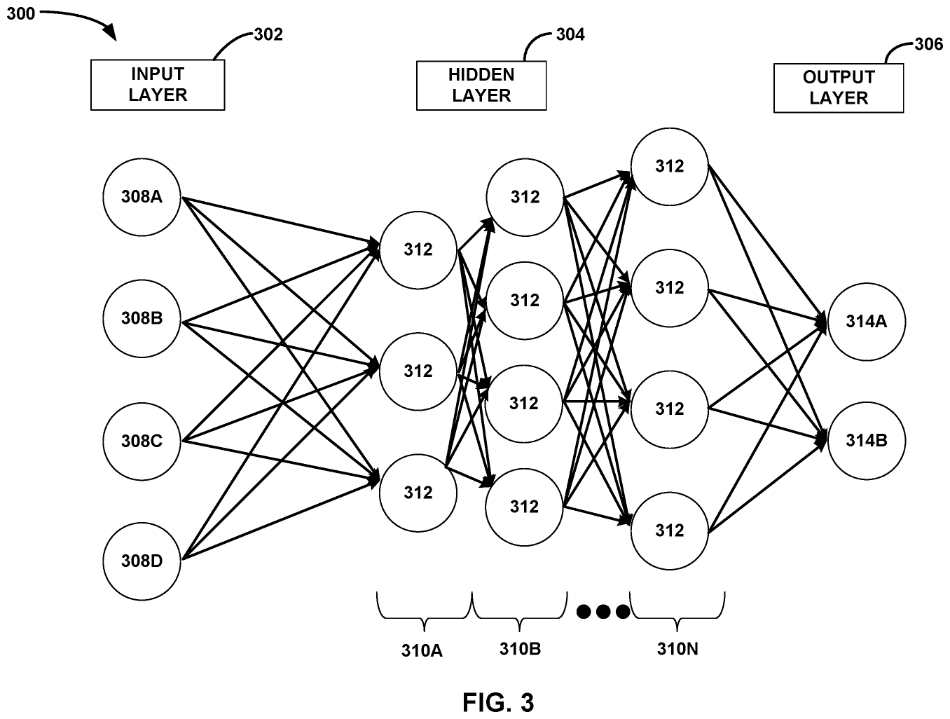

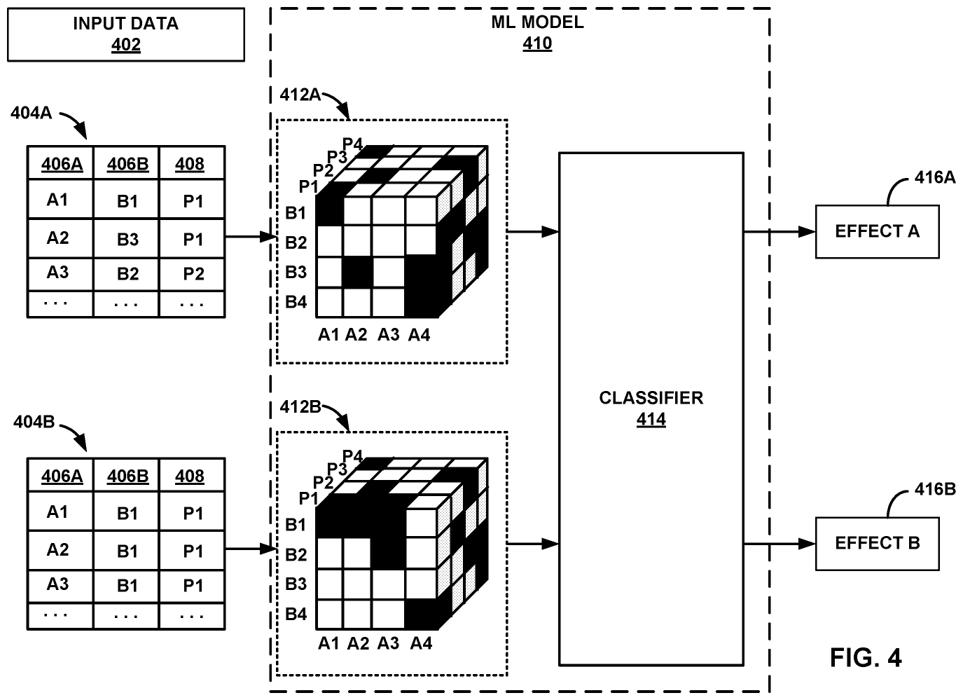

FIG. 3 is a conceptual diagram illustrating an example model configured to predict the startup conditions of IMD 104. While FIG. 3 describes the model as including neural network 300, other example models described herein may include other ML and/or non-ML techniques and models (e.g., a decision tree model, a support vector machine (SVM) model). Neural network 300 may include, but is not limited to, an artificial neural network (ANN) model and/or a distributed neural network (DNN) model. Neural network 300 may include an input layer 302, hidden layer 304, and an output layer 306. Neural network 300 may be stored in memory of one or more computing devices, computing systems, and/or cloud computing environments (e.g., in memory 112, in ML module 122). Processing circuitry 108 of external programmer 106 and/or processing circuitry 118 of computing system 116 may use the example process illustrated in FIG. 4 to input parameter values into a model and predict a likelihood of IMD 104 to encounter an unsuccessful startup attempt and/or a failure to start.

Input layer 302 includes inputs 308A-D (collectively referred to as “inputs 308”). Each of inputs 308 may represent a source of data input into neural network 300. In some examples, each of inputs 308 may represent a distinct retrieved parameter from IMD 104.

Input layer 302 may be connected to hidden layer 304 and inputs 308 may be transmitted to hidden layer 304. Hidden layer 304 may include layers 310A-N(collectively referred to as “layers 310”), each of layers 310 including one or more nodes 312. Hidden layer 304 may weigh and/or aggregate inputs 308 to produce an output (e.g., a predicted effect of a medical procedure) based on the input data. The structure of hidden layer 304 (e.g., number of layers 310, number of nodes 312, disposition of pathways between nodes 312) and/or the functions of hidden layer 304 (e.g., the manner of aggregation of inputs 308, the manner of weighing of inputs 308) may vary based on the desired output. In some examples, one or more computing devices, computing systems, and/or cloud computing environments (e.g., external programmer 106, computing system 116) may determine and/or adjust the number of layers 310, the structure of each layer 310, the weighing of each of inputs 308, and/or the manner of aggregation of inputs 308 via one or more ML training techniques.

Hidden layer 304 may determine, based on inputs 308 and functions performed by hidden layer 304, outputs 314A-B (collectively referred to as “outputs 314”) of output layer 306. Outputs 314 may include, but are not limited to, the probability that IMD 104 will experience an unsuccessful startup attempt or a failure to start. The probability may be represented as a percentage (e.g., 10% chance to fail, 10% chance IMD 104 will not fail) and/or as a Boolean (e.g., “YES” v. “NO”). For example, output 314A may indicate that there is a specific percentage chance that IMD 104 will experience an unsuccessful startup attempt or a failure to start and output 314B indicates a different percentage change. In some examples, output 314A indicates that IMD 104 will not experience an unsuccessful start or a failure to start and output 314B indicates that IMD 104 will experience an unsuccessful start or a failure to start.

FIG. 4 is a conceptual diagram illustrating an example process of inputting data into an example model for the prediction of the startup conditions of IMD 104. While FIG. 4 illustrated the example model as a ML model 410, other examples may include rule-based model (e.g., rule-based expert systems). Processing circuitry 108 of external programmer 106 and/or processing circuitry 118 of computing system 116 may use the example process illustrated in FIG. 4 to input parameter values into a model and predict a likelihood of IMD 104 to encounter an unsuccessful startup attempt and/or a failure to start.

Input data 402 for ML model 410 may be represented as multiway arrays 404A-B (collectively referred to as “multiway arrays 404”). Each of multiway arrays 404 may store a plurality of inputs 406A-B (collectively referred to as “inputs 406”). Each of inputs 406 may represent a parameter stored in IMD 104 and retrieved by external programmer 106. Each of multiway arrays 404 may store the values of inputs 406 and the position of the values of input 406 within a corresponding tensor representation of tensor representations 412A-B (collectively referred to as “tensor representations 412”).

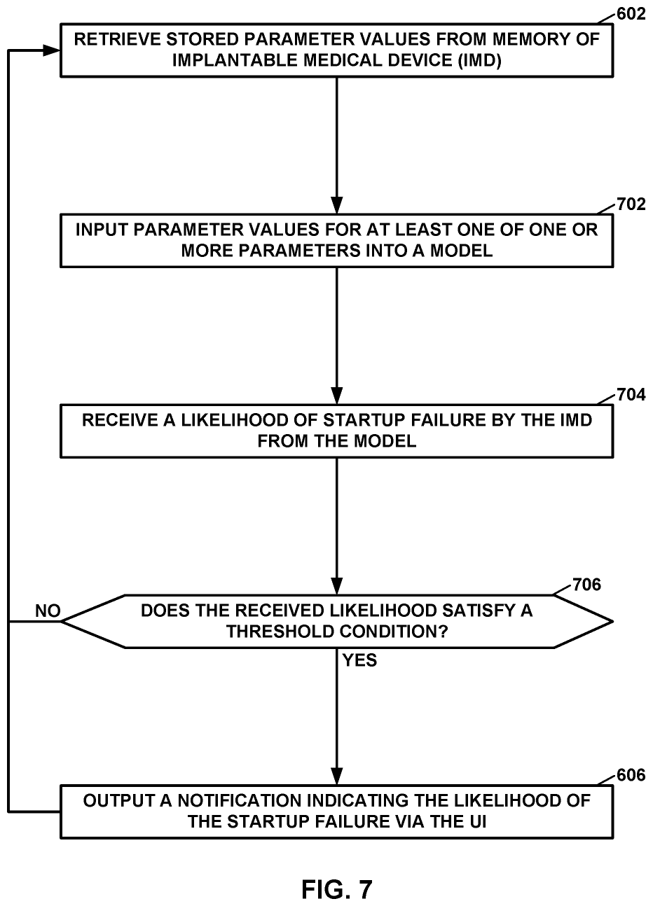

The position of particular values within the corresponding tensor representation 412 may be represented by the markers (e.g., markers A1-A4, B1-B4) and ports 408 stored in multiway arrays 404. Each of the plurality of markers may have a respective value of the feature represented by the corresponding input of inputs 406. Each of ports 408 may represent a point of data input (e.g., inputs 406) into classifier 414. For each combination of inputs 406A and 406B stored in multiway arrays 404, the corresponding tensor representation 412 may represent the combination of the values of the inputs 406 via a vector. As illustrated in FIG. 7, depending on the desired output, the combinations of inputs 406 and placements of the combinations of inputs 406 within tensor representation 412B may be different.

Multiway arrays 404 may be represented as tensor representations 412 which may be inputted into classifier 414 of ML model 710 to generate corresponding outputs 416A-B (collectively referred to as “outputs 416”). Each of outputs 416 may each represent a likelihood of occurrence of a particular outcome of a startup procedure (e.g., a successful startup attempt, an unsuccessful startup attempt, a failure to start). While the tensor representations 412 illustrated in FIG. 4 are third order tensors, other examples may include first order, second order, or fourth order or higher tensors as inputs to an example model.

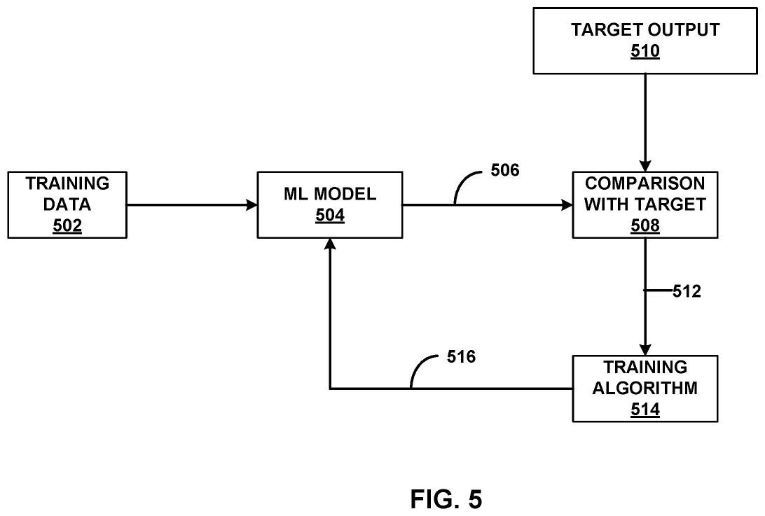

FIG. 5 is a block diagram illustrating an example process of training an example model for the prediction of the startup conditions of the IMD. While FIG. 5 is described primarily with reference to computing system 116, the example process described may be performed using one or more computing devices, computing systems, and/or cloud computing environments (e.g., external programmer 106 or the like). Computing system may generate ML model 504 (e.g., with randomly assigned weights and/or structure) and store ML model 504 in ML module 122 of memory 120 of computing system 116. ML model 504 may include, but is not limited to, a decision tree model, an ANN model, a DNN model, and/or a SVM model.

Computing system 116 may input training data 502 into ML model 504 to generate prediction 506. Training data 502 may include respective values for one or more parameters. The one or more parameters may include, but are not limited to, one or more of a number of startup attempts for each successful startup attempt for an IMD, a number of prior unsuccessful startup attempts by an IMD, a device identifier for an IMD, a device type for an IMD, an identifier for a patient implanted with an IMD, a timestamp of each startup attempt by an IMD, a duration of each startup attempt by an IMD, an amplitude of each electrical current delivered to an IMD during startup attempts, or a voltage of each electrical current delivered to an IMD during startup attempts. The respective values may be organized into training instances of a training set of training data 502. In some examples, each training instance may correspond to the respective parameter values for one or more startup attempts for an IMD. In some examples, each training instance may correspond to aggregated parameter values from a plurality of substantially similar IMDs (e.g., of the same device type and/or with similar device identifiers) which had undergone the startup procedure. In some examples, multiple training instances may correspond to a single IMD, each training instance corresponding to a different startup attempt. In some examples, computing system 116 generates one or more training instances of the set of training instances from parameter values retrieve from one or more IMDs, which may include or exclude IMD 104 and/or one or more IMDs that was prior implanted in patient 102.

Computing system 116 may perform comparison 508 between prediction 506 and target output 510. Target output 510 may include determined effects of startup procedures corresponding to the training instances of training data 502. Computing system 116 may determine, based on comparison 508, error data 512 between prediction 506 and target output 510. Computing system 116 may for each training instance in the training set, modify, based on particular parameter values and particular outcomes of startup procedures, ML model 504 to change a likelihood predicted by ML model 504 for the particular predicted outcome associated with the particular parameter values.

Based on the error data 512, computing system 116 may apply a training algorithm 514 (also referred to as “learning algorithm 514”) to adjust weights and/or structure of ML model 504. Computing system 116 may then transmit adjustments 516 to ML model 504 and adjust ML model 504 based on adjustments 516. Computing system 116 may then input subsequent data from training data 502 and perform the process until predictions 506 differ from target output 10 by less than a predetermined amount (e.g., by less than a predetermined percentage).

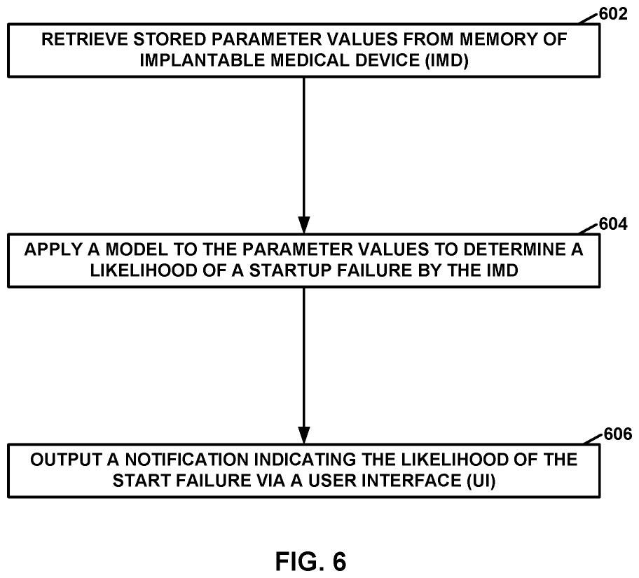

FIG. 6 is a flowchart illustrating an example process of predicting the startup conditions of IMD 104. While the example process illustrated in FIG. 6 is described below primarily with reference to system 100, the example process may be performed by computing devices, computing systems, or cloud computing environments of another medical device system described herein.

Processing circuitry 108 of external programmer 106 may retrieve stored parameter values from memory 212 of IMD 104 (602). As IMD 104 initiates startup procedures to power on the components of IMD 104, processing circuitry 202 may record, e.g., via sensor(s) 208, parameter values corresponding to each startup attempt (e.g., for each successful startup attempt, for each unsuccessful startup attempt). Processing circuitry 202 may store the parameter values in memory 212, e.g., in parameter value(s) module 214.

Processing circuitry 108 may retrieve at least a portion of the stored parameter values from IMD 104 via communications circuitry 110. In some examples, processing circuitry 108 transmits, via communications circuitry 110, instructions to IMD 104 to cause IMD 104 to transmit the parameter values to external programmer 106, e.g., via communications circuitry 204. In some examples, IMD 104 automatically outputs the parameter values to external programmer 106 (e.g., after the conclusion of the startup procedure, periodically). Processing circuitry 108 may store the retrieved parameter values in memory 112 of external programmer 106. Processing circuitry may transmit the retrieved parameter values to computing system 116 via communications circuitry 110. Computing system 116 may subsequently store the parameter values in memory 120 of computing system 116.

Communication circuitry 110 may communicate with IMD 104 and/or computing system 116 via wired communication or by wireless communication techniques. Wireless communication techniques may include radiofrequency (RF) communication techniques, e.g., via an antenna (not shown). Communication circuitry 204 may include a radio transceiver configured for communication according to standards of protocols, such as 3G, 4G, 5G, WiFi (e.g., 802.11 or 802.15 ZigBee), Bluetooth®, or Bluetooth® Low Energy (BLE).

Processing circuitry 108 may apply a model (e.g., ML model 300, 410, 504) to the retrieve parameter values to determine a likelihood of startup failure by IMD 104 (604). Processing circuitry 108 may retrieve and execute instructions for the model, e.g., from memory 112, from memory 120. The model may be trained (e.g., as previously described herein) to receive value(s) for one or more parameters and determine a likelihood that IMD 104 will experience an unsuccessful startup attempt and/or a failure to start based on the inputted value(s). The value(s) and/or the combination of values may be determined by processing circuitry 118 during the training of the model. Processing circuitry 108 may input retrieved parameter values from IMD 104 into the model based on the required input value(s) for the model.

Processing circuitry 108 may determine, based on the output of the model, the probability and/or likelihood that IMD 104 experiences an unsuccessful startup attempt and/or a failure to start. The likelihood may be represented as a percentage chance (e.g., of an unsuccessful startup attempt, of a successful startup attempt, of a failure to start), a Boolean (e.g., “YES” or “NO” to whether IMD 104 will experience an unsuccessful startup attempt and/or a failure to start), and/or any other representations of a probability (e.g., a ratio). In some examples, processing circuitry 108 determines, based on the output of the model, one or more causes for a future unsuccessful startup attempt and/or failure to start. In some examples, processing circuitry 108 may input retrieved parameter values to the model and/or one or more other models to determine an expected timeframe for an unsuccessful startup attempt and/or a failure to start.

The model may determine the probability of IMD 104 experiencing a startup failure within a period of time (e.g., within an expected lifespan of IMD 104). In some examples, the period of time includes, a period of time for which patient 102 is expected to receive IMD 104 and/or another IMD of system 100. The period of time may be up to one year or up to several years (e.g., up to ten years, up to twenty years).

Processing circuitry 108 may output a notification indicating the likelihood of the startup failure via UI 114 (606). The notification may include, but is not limited, the likelihood of an unsuccessful startup attempt and/or a failure to start, one or more potential causes of the unsuccessful startup attempt and/or failure to start, an expected timeframe for the unsuccessful startup attempt and/or failure to start, and/or recommended adjustments to the startup procedure of IMD 104 to reduce the likelihood of the unsuccessful startup attempt and/or failure to start. UI 114 may output the notification as one or more of a visual signal or an auditory signal. In some examples, processing circuitry 108 may transmit, via communications circuitry 110, the notification to one or more computing devices, computing systems (e.g., computing system 116), and/or cloud computing environments in communication with external programmer 106.

One or more of processing circuitry 108, 118, may adjust one or more parameters of a startup procedure for IMD 104 based on the determined likelihood of startup failure. Processing circuitry 108 and/or 118 may adjust the startup procedure (e.g., adjust one or more parameters for the startup procedure) for IMD 104 to, e.g., to increase a likelihood that IMD 104 will successfully start up. Parameters for the startup procedure may include, but are not limited to, an amplitude of an electrical signal or current transmitted to IMD 104 to start motor 207 of IMD 104, a voltage of an electrical signal or current transmitted to IMD 104 to start motor 207 of IMD 104, a startup speed for rotors 206 and/or motor 207, a number and/or selection of components within IMD 104 to be powered on during startup of IMD 104, an order of components to be powered on during startup of IMD 104, or a number of startup attempts during startup of IMD 104. For example, processing circuitry 108 may increase, e.g., based on a determination of the likelihood of the startup failure exceeding a threshold likelihood, increase an amplitude and/or a voltage of the electrical current to be transmitted to IMD 104 during the startup procedure.

In some examples, the adjusted startup procedure parameters are already be stored in IMD 104 and toggled on by the instruction from processing circuitry 108 and/or 118. In some examples, the adjusted startup procedure parameters are transmitted to IMD 104 by processing circuitry 108 and/or 118 via communications circuitry 110 of external programmer 106.

FIG. 7 is a flowchart illustrating another example process of predicting and outputting the startup conditions of IMD 104. While the example process illustrated in

FIG. 7 is described below primarily with reference to processing circuitry 108 system 100, the example process may be performed by other computing devices, computing systems, or cloud computing environments of system 100 (e.g., processing circuitry 118 of computing system 116) and/or another medical device system described herein.

Processing circuitry 108 may retrieve stored parameter values from memory 212 of IMD 104 (602) and may input parameter values for at least one of the one or more parameters into a model (702). The model may include, but is not limited to, a ML model as previously described herein. The model may be trained to receive parameter values for one or more parameters stored in IMD 104 and to output a likelihood that IMD 104 will experience a startup failure (e.g., an unsuccessful startup attempt and/or a failure to start) based on the inputted values. The specific parameter(s) required as inputs by the model may be determined, (e.g., by computing system 116) during training of the model.

Processing circuitry 108 may receive a likelihood of startup failure by IMD 104 from the model (704). Processing circuitry 108 may receive the likelihood as an output from the model. The likelihood may be represented as, but is not limited to, a percentage probability or a Boolean. In some examples, processing circuitry 108 may receive, from the model and/or one or more other models, a predicted cause of the startup failure, a timeframe for the startup failure, and/or adjustments to a startup procedure of IMD 104 to mitigate and/or eliminate the risk of the startup failure.

Processing circuitry 108 may determine whether the received likelihood of startup failure satisfies a threshold condition (706). The threshold condition may represent a threshold likelihood that IMD 104 will experience a startup failure. The threshold likelihood may be represented as, but is not limited to, a threshold percentage (e.g., up to 5%, up to 10%) or a threshold Boolean. In some examples, processing circuitry 108 determines that the threshold condition is satisfied based on a determination that the received likelihood is less than or equal to the threshold percentage (e.g., that there is less than the threshold percentage chance that IMD 104 will not experience a startup failure). In some examples, processing circuitry 108 determines that the threshold condition is satisfied based on a determination that the received likelihood is greater than or equal to the threshold percentage (e.g., that there is more than the threshold percentage chance that IMD 104 will experience a startup failure. In some examples, processing circuitry 108 determines that the threshold condition is satisfied based on a determination that the received Boolean does or does not match the threshold Boolean.

Based on a determination that the received likelihood does not satisfy the threshold condition (“NO” branch of 706), processing circuitry 108 may continue to retrieve stored parameter values from memory 212 of IMD 104 (602) and perform steps 602, and 702-704 to monitor for any changes in the likelihood that IMD 104 will experience a startup failure. Based on a determination that the received likelihood satisfies the threshold condition (“YES” branch of 706), processing circuitry 108 may output a notification indicating the likelihood of startup failure via UI 114 (606).

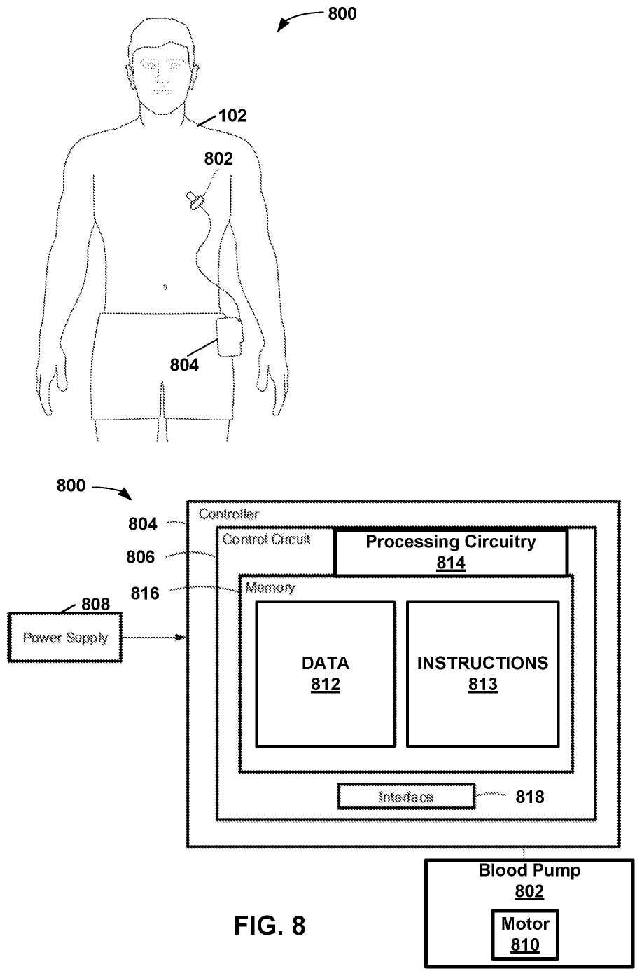

FIG. 8 is a conceptual diagram of a medical device system 800 for predicting startup conditions of an implantable blood pump device 802. Medical device system 800 (also referred to herein as “system 800”) may be an example of system 100 and blood pump device 802 may be an example of IMD 104. Blood pump device 802 may be implanted within patient 102 (e.g., in a heart of patient 102) and may be coupled to a controller 804 external to the body of patient 102.

Blood pump device 802 may be entirely or partially implanted within patient 102. Blood pump device 802 may include a movable element (e.g., a rotor) configured to pump blood from the heart of patient 102 to the rest of the body of patient 102. Blood pump device 802 may be a variable speed blood pump configured to dynamically change speeds, e.g., under the control of processing circuitry 814 of controller 804). Blood pump device 802 may be in communication with controller 804. Controller 804 may be positioned external to patient 102 or may be implanted within patient 102 and may be in either wired or wireless communication with blood pump device 102. Controller 804 may include control circuit 806 which includes power supply 808, processing circuitry 814, memory 816, and interface 818. Blood pump device 802 may include motor 810. Processing circuitry 814 may be substantially similar to processing circuitry 108 or processing circuitry 118, except as described below.

Determination of the likelihood of startup failure based on parameter values stored in system 800 may be performed by processing circuitry of blood pump device 802 and/or processing circuitry 814 of controller 804. Parameter values may be stored in memory 816 (e.g., in data module 812 of memory 816). Controller 804 may monitor parameter values associated with each startup attempt by blood pump device 802 and store the determined parameter values in memory 816. For example, controller 804 may monitor the voltage and/or amplitude of each electrical current transmitted from power supply 808 to blood pump device 802 to attempt to start motor 810.

Processing circuitry 814 may retrieve and execute instructions stored in instructions module 813 of memory 816 to apply an algorithm (e.g., one or more ML models, rule-based models and/or algorithms) to the parameter values stored in data module 812 to determine a likelihood that blood pump device 802 will experience a startup failure (e.g., within a threshold period of time). Processing circuitry 814 may input parameter values stored in data module 812 into the algorithm (e.g., into one or more ML models) to determine a likelihood that blood pump device 802 will experience a startup failure, e.g., as previously described herein.

Controller 804 may output the determined likelihood (e.g., to patient 102, to a clinician) through a notification presented via interface 818. Interface 818 may present the notification via a visual or audio output signal. In some examples, processing circuitry 814 may adjust, based on the determine likelihood, parameters for a startup procedure for blood pump device 802. The parameters for the startup procedure may be stored in instructions module 813 of memory 816 of controller 804.

If blood pump device 802 experiences a startup failure, blood pump device 802 may be unable to pump blood through the heart and/or circulatory system of patient 102. Combinations of parameters from blood pump device 802, e.g., as previously described herein, may correlate with and/or indicate a likelihood that blood pump device 802 will experience a future startup failure. The combinations of parameters may involve complex interactions with multiple independent and/or unrelated parameters, which would make it infeasible for a clinician to evaluate the likelihood of a future startup failure based on the parameter data. Applying the algorithm (e.g., a ML model) to the values for the one or more parameters may allow for a rapid and accurate analysis of the complex parameter data to effectively predict the likelihood of blood pump device 802 will experience a startup failure. The accurate and rapid prediction of the likelihood of startup failure may allow for the replacement of blood pump device 802 and/or adjustments in the startup procedure of blood pump device 802 to reduce a risk of an inability by blood pump device 802 to pump blood through the heart and/or circulatory system of patient 102.

Controller 804, control circuitry 806, and processing circuitry 814 may include various type of hardware, including, but not limited to, microprocessors, controllers, DSPs, ASICS, FPGAs, or equivalent discrete or integrated logic circuitry, as well as combinations of such components. The term “processor,” “processing circuitry,” “controller,” and “control circuitry” may generally refer to any of the foregoing logic circuitry, alone or in combination with other logic circuitry, or any other equivalent circuitry. Controller 804, control circuitry 806, and processing circuitry 814 may represent hardware that can be configured to implement firmware and/or software that sets forth one or more algorithms described herein. For example, processing circuitry 814 can include processing circuitry configured to implement functionality, process instructions, or both for execution within controller 804 of processing instructions stored within memory 816.

Memory 816 may store program instructions, such as software, which may include one or more program modules, which are executable by circuitry of controller 804. When executed by circuitry, such program instructions may cause controller 804 to provide the functionality ascribed to controller 804 herein. The program instructions may be embodied in software and/or firmware. Memory 816 may include any volatile, non-volatile, magnetic, optical, or electrical media, such as RAM, ROM, NVRAM, EEPROM, flash memory, or any other digital media.

Interface 818 may include any suitable user interface device. Interface 818 may be configured to generate an output through tactile, audio, or video sources and/or receive inputs from a user. Interface 818 may include sound generating circuitry, a display, a mouse, a button, a keyboard, a voice responsive system, video camera, microphone, touchscreen, or any other type of device for detecting a command from a user.

The techniques of this disclosure may be implemented in a wide variety of computing devices, medical devices, or any combination thereof. Any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to various modules and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated, discrete logic circuitry, or other processing circuitry, as well as any combinations of such components, remote servers, remote client devices, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. For example, any module described herein may include electrical circuitry configured to perform the features attributed to that particular module, such as fixed function processing circuitry, programmable processing circuitry, or combinations thereof.

The techniques described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

It should be noted that medical device system 100, and the techniques described herein, may not be limited to use in a human patient. In alternative examples, medical device system 100 may be implemented in non-human patients, e.g., primates, canines, equines, pigs, and felines. These other animals may undergo clinical or research therapies that my benefit from the subject matter of this disclosure. Various examples are described herein, such as the following examples.

Example 1: a system comprising: an implantable medical device (IMD) comprising: an impeller configured to cause blood flow in a patient; a first memory; parameter sampling circuitry configured to determine values for one or more parameters associated with a startup of the implantable blood pump; and first processing circuitry configured to store, in the first memory, the values for one or more parameters with corresponding timestamps; and a programming device comprising: communications circuitry; a second memory; a user interface (UI); and processing circuitry configured to: retrieve, from the IMD and via the communications circuitry, the values for the one or more parameters associated with the startup of the IMD; apply a machine learning (ML) model stored in the memory to the values for the one or more parameters, wherein the ML model is trained via a data set comprising a plurality of parameter values from one or more IMDs and corresponding results of startup procedures performed by the one or more IMDs; determine, based on an output from the ML model, a probability of the IMD having a future startup failure within a period of time, wherein blood is not pumped by the IMD after an occurrence of a startup failure; and cause the UI to output a notification indicating the probability of the IMD experiencing the future startup failure.

Example 2: the system of example 1, wherein the IMD comprises an implantable blood pump.

Example 3: the system of any of examples 1 or 2, wherein the processing circuitry is further configured to transmit, via the communications circuitry and based on the probability of the IMD experiencing a startup failure within the period of time, one or more instructions to adjust a startup procedure of the IMD.

Example 4: the system of example 3, wherein the one or more instructions to adjust the startup procedure of the IMD comprises one or more instructions to adjust one or more of an amplitude or a voltage of an electric current transmitted from a power source to the IMD to start the IMD.

Example 5: the system of example 4, wherein the one or more instructions to adjust one or more of the amplitude or a voltage of the electric current comprises: one or more instructions to increase one or more of the amplitude or the voltage of the electric current.

Example 6: the system of any of examples 3-5, wherein the one or more instructions to adjust the startup procedure of the IMD comprises one or more instructions to adjust a starting speed of an impeller of the IMD.

Example 7: the system of any of examples 3-6, wherein the one or more instructions to adjust the startup procedure of the IMD comprises one or more instructions to adjust a number of startup attempts to start the IMD within the startup procedure.

Example 8: the system of any of examples 1-7, wherein the one or more parameters comprise one or more of: an amplitude of an electric current required to start the IMD; a voltage of the electric current required to start the IMD; a number of startup attempts required by the IMD to start the IMD; a time of one or more prior successful startup attempts by the IMD; an indication of a prior failure to start by the IMD; a device type of the IMD; or a device identifier of the IMD.

Example 9: the system of any of examples 1-8, wherein to determine the probability of the IMD experiencing a future startup failure, the processing circuitry is configured to determine a percentage chance of the IMD experiencing a failure to start within the period of time.

Example 10: the system of any of examples 1-9, wherein to determine the probability of the IMD experiencing a future startup failure, the processing circuitry is configured to determine a Boolean indicator indicating whether the IMD will experience a failure to start within the period of time.

Example 11: the system of any of examples 1-10, wherein the one or more IMDs comprises one or more other IMDs different from the IMD.

Example 12: the system of any of examples 1-11, wherein the processing circuitry is further configured to: retrieve, via the communications circuitry and from a network, instructions for the ML model from a computing device; and execute the retrieved instructions to apply the ML model to the parameter values.

Example 13: the system of any of examples 1-12, wherein the ML model comprises one or more of: a decision tree model; an artificial neural network (ANN) model; a distributed neural network (DNN) model; or a support vector machine (SVM) model.

Example 14: a computing device comprising: communications circuitry; memory; a user interface (UI); and processing circuitry configured to: retrieve, via communications circuitry and from an implantable medical device (IMD) implanted in a patient, values for one or more parameters associated with a startup of the IMD; apply a machine learning (ML) model stored in the memory to the parameter values, wherein the ML model is trained via a data set comprising a plurality of parameter values from one or more IMDs and corresponding results of startup procedures performed by the one or more IMDs; determine, based on outputs from the ML model, a probability of the IMD having a future startup failure within a period of time; and cause the UI to output a notification indicating the probability of the IMD experiencing a future startup failure.

Example 15: the computing device of example 14, where the IMD comprises an implantable blood pump.

Example 16: the computing device of any of examples 14 and 15, wherein the processing circuitry is configured to: receive, via the communications circuitry, the data set; and train the ML model via the data set.

Example 17: the computing device of any of examples 14-16, wherein the processing circuitry is further configured to transmit, via the communications circuitry and based on the probability of the IMD experiencing a future startup failure, one or more instructions to adjust a startup procedure of the IMD.

Example 18: the computing device of example 17, wherein the one or more instructions to adjust the startup procedure of the IMD comprises one or more instructions to adjust one or more of an amplitude or a voltage of an electric current transmitted from a power source to the IMD to start the IMD.

Example 19: the computing device of example 18, wherein the one or more instructions to adjust one or more of the amplitude or a voltage of the electric current comprises: one or more instructions to increase one or more of the amplitude or the voltage of the electric current.

Example 20: the computing device of any of examples 17-19, wherein the one or more instructions to adjust the startup procedure of the IMD comprises one or more instructions to adjust a starting speed of an impeller of the IMD.

Example 21: the computing device of any of examples 17-20, wherein the one or more instructions to adjust the startup procedure of the IMD comprises one or more instructions to adjust a number of startup attempts to start the IMD within the startup procedure.

Example 22: the computing device of any of examples 14-21, wherein the one or more parameters comprise one or more of: an amplitude of an electric current required to start the IMD; a voltage of the electric current required to start the IMD; a number of startup attempts required by the IMD to start the IMD; a time of one or more prior successful startup attempts by the IMD; an indication of a prior failure to start by the IMD; a device type of the IMD; or a device identifier of the IMD.

Example 23: the computing device of any of examples 14-22, wherein to determine a probability of the IMD experiencing a future startup failure, the processing circuitry is configured to determine a percentage chance of the IMD experiencing a failure to start within the period of time.

Example 24: the computing device of any of examples 14-22, wherein to determine a probability of the IMD experiencing a future startup failure, the processing circuitry is configured to determine a Boolean indicator indicating whether the IMD will experience a failure to start within the period of time.

Example 25: the computing device of any of examples 14-24, wherein the processing circuitry is further configured to: retrieve, via the communications circuitry and from a network, instructions for the ML model from a computing device; and execute the retrieved instructions to apply the ML model to the parameter values.

Example 26: the computing device of any of examples 14-25, wherein the ML model comprises one or more of: a decision tree model; an artificial neural network (ANN) model; a distributed neural network (DNN) model; or a support vector machine (SVM) model.

Example 27: a computer-readable medium comprising instructions that, when executed, causes processing circuitry of a computing device to: retrieve, via communications circuitry of the computing device and from an implantable medical device (IMD) implanted in a patient, values for one or more parameters associated with a startup of the IMD; apply a machine learning (ML) model to the parameter values, wherein the ML model is trained via a data set comprising a plurality of parameter values from one or more IMDs and corresponding results of startup procedures performed by the one or more IMDs;

determine, based on outputs from the ML model, a probability of the IMD having a future startup failure within a period of time; and cause a user interface (UI) of the computing device to output a notification indicating the probability of the IMD experiencing a future startup failure within the period of time.

Example 28: the computer-readable medium of example 27, further comprising instructions that, when executed, causes the processing circuitry to: transmit, via the communications circuitry and based on the probability of the IMD experiencing a future startup failure, one or more instructions to adjust a startup procedure of the IMD.

Example 29: the computer-readable medium of example 28, wherein the one or more instructions to adjust the startup procedure of the IMD comprises one or more instructions to adjust one or more of an amplitude or a voltage of an electric current transmitted from a power source to the IMD to start the IMD.

Example 30: the computer-readable medium of example 29, wherein the one or more instructions to adjust one or more of the amplitude or a voltage of the electric current comprises: one or more instructions to increase one or more of the amplitude or the voltage of the electric current.

Example 31: the computer-readable medium of any of examples 28-30, wherein the one or more instructions to adjust the startup procedure of the IMD comprises one or more instructions to adjust a starting speed of an impeller of the IMD.

Example 32: the computer-readable medium of any of examples 28-31, wherein the one or more instructions to adjust the startup procedure of the IMD comprises one or more instructions to adjust a number of startup attempts to start the IMD within the startup procedure.

Example 33: the computer-readable medium of any of examples 27-32, wherein the IMD comprises an implantable blood pump.

Example 34: the computer-readable medium of any of examples 27-33, wherein the one or more parameters comprise one or more of: an amplitude of an electric current required to start the IMD; a voltage of the electric current required to start the IMD; a number of startup attempts required by the IMD to start the IMD; a time of one or more prior successful startup attempts by the IMD; an indication of a prior failure to start by the IMD; a device type of the IMD; or a device identifier of the IMD.

Example 35: the computer-readable medium of any of examples 27-34, further comprising instructions that, when executed, causes the processing circuitry to determine the probability of the IMD experiencing a future startup failure by determining a percentage chance of the IMD experiencing a failure to start in the future within the period of time.

Example 36: the computer-readable medium of any of examples 27-35, further comprising instructions that, when executed, causes the processing circuitry to determine the probability of the IMD experiencing a future startup failure by determining a Boolean indicator indicating whether the IMD will experience a failure to start within the period of time.

Example 37: the computer-readable medium of any of examples 27-36, further comprising instructions that, when executed, causes the processing circuitry to: retrieve, via the communications circuitry and from a network, instructions for the ML model from a computing device; and execute the retrieved instructions to apply the ML model to the parameter values.

Example 38: the computer-readable medium of any of examples 27-37, wherein the ML model comprises one or more of: a decision tree model; an artificial neural network (ANN) model; a distributed neural network (DNN) model; or a support vector machine (SVM) model.

Example 39: a system comprising: an implantable blood pump comprising: a pump housing; an impeller disposed within the pump housing, and a motor disposed within the pump housing and coupled to the impeller, wherein the motor is configured to rotate the impeller within the pump housing to pump blood through the implantable blood pump; a power supply; and a controller coupled to the implantable blood pump and to the power supply, the controller comprising: a user interface (UI), a memory, and a processing circuitry configured to: cause the power supply to transmit an electrical signal to the motor of the implantable blood pump to start the implantable blood pump; retrieve from one or more of the impeller, the motor, or the power supply, values for the one or more parameters; apply a machine learning (ML) model stored in the memory to the values for the one or more parameters, wherein the ML model is trained via a data set comprising a plurality of parameter values from one or more implantable blood pumps and corresponding results of startup procedures performed by the one or more implantable blood pumps; determine, based on an output from the ML model, a probability of the implantable blood pump having a future startup failure within a period of time, wherein blood is not pumped by the implantable blood pump after an occurrence of a startup failure; and cause the UI to output a notification indicating the probability of the implantable blood pump experiencing the future startup failure.

Various examples have been described herein. Any combination of the described operations or functions is contemplated. These and other examples are within the scope of the following claims.

What is claimed is:

1. A system comprising:
an implantable medical device (IMD) comprising:
an impeller configured to cause blood flow in a patient;
a first memory;
parameter sampling circuitry configured to determine values for one or more parameters associated with a startup of the implantable blood pump; and
first processing circuitry configured to store, in the first memory, the values for one or more parameters with corresponding timestamps; and
a programming device comprising:
communications circuitry;
a second memory;
a user interface (UI); and
processing circuitry configured to:
retrieve, from the IMD and via the communications circuitry, the values for the one or more parameters associated with the startup of the IMD;
apply a machine learning (ML) model stored in the memory to the values for the one or more parameters, wherein the ML model is trained via a data set comprising a plurality of parameter values from one or more IMDs and corresponding results of startup procedures performed by the one or more IMDs;
determine, based on an output from the ML model, a probability of the IMD having a future startup failure within a period of time, wherein blood is not pumped by the IMD after an occurrence of a startup failure; and
cause the UI to output a notification indicating the probability of the IMD experiencing the future startup failure.

2. The system of claim 1, wherein the IMD comprises an implantable blood pump.

3. The system of claim 1, wherein the processing circuitry is further configured to transmit, via the communications circuitry and based on the probability of the IMD experiencing a startup failure within the period of time, one or more instructions to adjust a startup procedure of the IMD.

4. The system of claim 3, wherein the one or more instructions to adjust the startup procedure of the IMD comprises one or more instructions to adjust one or more of an amplitude or a voltage of an electric current transmitted from a power source to the IMD to start the IMD.

5. The system of claim 4, wherein the one or more instructions to adjust one or more of the amplitude or a voltage of the electric current comprises:
one or more instructions to increase one or more of the amplitude or the voltage of the electric current.

6. The system of claim 3, wherein the one or more instructions to adjust the startup procedure of the IMD comprises one or more instructions to adjust a starting speed of an impeller of the IMD.

7. The system of claim 3, wherein the one or more instructions to adjust the startup procedure of the IMD comprises one or more instructions to adjust a number of startup attempts to start the IMD within the startup procedure.

8. The system of claim 1, wherein the one or more parameters comprise one or more of:
an amplitude of an electric current required to start the IMD;
a voltage of the electric current required to start the IMD;
a number of startup attempts required by the IMD to start the IMD;
a time of one or more prior successful startup attempts by the IMD;
an indication of a prior failure to start by the IMD;
a device type of the IMD; or
a device identifier of the IMD.

9. The system of claim 1, wherein to determine the probability of the IMD experiencing a future startup failure, the processing circuitry is configured to determine a percentage chance of the IMD experiencing a failure to start within the period of time.

10. The system of claim 1, wherein to determine the probability of the IMD experiencing a future startup failure, the processing circuitry is configured to determine a Boolean indicator indicating whether the IMD will experience a failure to start within the period of time.

11. The system of claim 1, wherein the processing circuitry is further configured to:
retrieve, via the communications circuitry and from a network, instructions for the ML model from a computing device; and
execute the retrieved instructions to apply the ML model to the parameter values.

12. The system of claim 1, wherein the ML model comprises one or more of:
a decision tree model;
an artificial neural network (ANN) model;
a distributed neural network (DNN) model; or
a support vector machine (SVM) model.

13. A computing device comprising:
communications circuitry;
memory;
a user interface (UI); and
processing circuitry configured to:
retrieve, via communications circuitry and from an implantable medical device (IMD) implanted in a patient, values for one or more parameters associated with a startup of the IMD;
apply a machine learning (ML) model stored in the memory to the parameter values, wherein the ML model is trained via a data set comprising a plurality of parameter values from one or more IMDs and corresponding results of startup procedures performed by the one or more IMDs;
determine, based on outputs from the ML model, a probability of the IMD having a future startup failure within a period of time; and
cause the UI to output a notification indicating the probability of the IMD experiencing a future startup failure.

14. The computing device of claim 13, where the IMD comprises an implantable blood pump.

15. The computing device of claim 13, wherein the processing circuitry is configured to:
receive, via the communications circuitry, the data set; and
train the ML model via the data set.

16. The computing device of claim 13, wherein the processing circuitry is further configured to transmit, via the communications circuitry and based on the probability of the IMD experiencing a future startup failure, one or more instructions to adjust a startup procedure of the IMD.

17. The computing device of claim 16, wherein the one or more instructions to adjust the startup procedure of the IMD comprises one or more instructions to adjust one or more of an amplitude or a voltage of an electric current transmitted from a power source to the IMD to start the IMD.

18. The computing device of claim 17, wherein the one or more instructions to adjust one or more of the amplitude or a voltage of the electric current comprises:
one or more instructions to increase one or more of the amplitude or the voltage of the electric current.

19. The computing device of claim 16, wherein the one or more instructions to adjust the startup procedure of the IMD comprises:
one or more instructions to adjust a starting speed of an impeller of the IMD; and/or
one or more instructions to adjust a number of startup attempts to start the IMD within the startup procedure.

20. A system comprising:
an implantable blood pump comprising:
a pump housing;
an impeller disposed within the pump housing, and
a motor disposed within the pump housing and coupled to the impeller, wherein the motor is configured to rotate the impeller within the pump housing to pump blood through the implantable blood pump;
a power supply; and
a controller coupled to the implantable blood pump and to the power supply, the controller comprising:
a user interface (UI),
a memory, and
a processing circuitry configured to:
cause the power supply to transmit an electrical signal to the motor of the implantable blood pump to start the implantable blood pump;
retrieve from one or more of the impeller, the motor, or the power supply, values for the one or more parameters;
apply a machine learning (ML) model stored in the memory to the values for the one or more parameters, wherein the ML model is trained via a data set comprising a plurality of parameter values from one or more implantable blood pumps and corresponding results of startup procedures performed by the one or more implantable blood pumps;
determine, based on an output from the ML model, a probability of the implantable blood pump having a future startup failure within a period of time, wherein blood is not pumped by the implantable blood pump after an occurrence of a startup failure; and
cause the UI to output a notification indicating the probability of the implantable blood pump experiencing the future startup failure.
